(19) United States
(12) Patent Application Publication
(10) Pub. No.: US 2013/0316389 A1
(43) Pub. Date: Nov. 28, 2013

(54) Method and System for Determining Characteristics of an Embryo and Uses Thereof

(71) Applicants: Oliver Tills, Plymouth (GB); Simon Rundle, Ivybridge (GB); Phil Culvershouse, Callington (GB); John Spicer, Plymouth (GB); Tabitha Silja Bitterli, Plymouth (GB)

(72) Inventors: Oliver Tills, Plymouth (GB); Simon Rundle, Ivybridge (GB); Phil Culvershouse, Callington (GB); John Spicer, Plymouth (GB); Tabitha Silja Bitterli, Plymouth (GB)

(73) Assignee: University of Plymouth, Plymouth, Devon (GB)

(21) Appl. No.: 13/877,318

(22) PCT Filed: Sep. 30, 2011
(86) PCT No.: PCT/GB2011/001434

§ 371 Date: Jul. 8, 2013

(30) Foreign Application Priority Data
Oct. 2, 2010 (GB) 1016616.3

(51) Int. Cl. G01N 33/483

(52) U.S. Cl. CPC G01N 33/4833
USPC 435/29; 435/288.7

(57) ABSTRACT

A method for characterising an embryo is provided, the method comprising determining characteristics of the pattern of movement of the embryo; and comparing the determined characteristics with reference data relating to the expected movement pattern of the subject embryo. The method is of use in the staging of embryos and in analysing an environment to which the embryo has been exposed. A system for characterising an embryo is also provided.

The present invention relates to a method for determining characteristics of embryos and to a system for performing the same. The present invention further relates to uses of the method and system.

The determination of the developmental stage of live embryonic animals is a frequently applied technique. Such staging of embryos is a key tool in the study of the development of animals and provides a more general tool for biological studies. The staging of embryos may be applied to embryos that are free living, for example the embryos of shrimps and some fish, as well as to embryos contained within an egg capsule, such as some crustaceans, molluscs and lower vertebrate species. For a discussion of the techniques and use of embryo staging, reference is made to ‘A history of normal plates, tables and stages in vertebrate embryology’, Hopwood, N., International Journal of Developmental Biology, 51 (2007), pages 1 to 26. The analysis and staging of embryos of higher organisms, such as mammals, may also be possible, using suitable imaging techniques.

The staging of embryos to determine their development is carried out manually. In particular, the embryos are visually inspected and the observations compared with published ‘normal’ tables of embryo development for the species of animal being observed. An example of such normal tables are disclosed in ‘Normal table of Xenopus laevis’, Nieuwkoop, P. D. et at., (Daudin), North-Holland Publishing Companay, Amsterdam (1956), and ‘Normal table of postembryonic zebrafish development: staging by externally visible anatomy of the living fish’, Parichy, D. M. et al, Developmental Dynamics 238 (2009). However, the manual analysis of embryos is very time consuming. The task requires a high degree of attention from the observer and is challenging to carry out for extended periods of time. Further, the analysis relies upon the discretion of the observer, in particular when comparing the observed embryos with the data contained in the normal tables being referenced. As a result, there is considerable debate as to the efficacy of manual staging and the meaning of the results obtained.

Accordingly, there is a need for an improved method of determining the characteristics of embryos, in particular their developmental stage. In particular, it would be especially advantageous if the staging of embryos could be automated.

The application of image analysis techniques to the analysis of the biological development of organisms is known in the art. For example, the imaging analysis of plant development is described in ‘The use of image analysis and automation for mitotic index in apical conifer meristems’, Sundblad, L.-G., et al., Journal of Experimental Botany, 49 (1998), pages 1749 to 1756. Further, the image analysis of Drosophila is described in ‘Towards an image analysis toolbox for high-throughput Drosophila embryo RNAi screens’, Kellogg, R. A., et al., 4^{th} IEEE International Symposium on Biomedical Imaging: from Nano to Macro, (2007). However, none of these techniques is applicable to the analysis of living embryos in real time.

EP 0 162 688 discloses an environment monitoring system. The system monitors the movements of a plurality of living organisms exposed to the environment under investigation. The movement of the organisms may be continuous or periodic. The system further measures selected parameters of the environment, such as temperature, the results of which measurements are used to generate a predicted movement pattern of the organisms. The measured movements of the organisms are compared with the predicted movement patterns and an alarm or warning generated when the predicted and observed movement patterns are different.

EP 1 852 058 is concerned with a system for monitoring fetus movement and a fetus movement information collecting device. The system comprises an electrical capacitance sensor for detecting movement of the fetus, which in use is placed on the abdomen of the mother. Signals from the fetal movement sensor are transmitted to a processor to determine patterns of movement of the fetus. The results of the movement pattern determination are displayed for viewing.

A system and method for determining lameness in an animal, such as a horse, is described in WO 2008/011590. The system comprises a plurality of motion sensors attached to various parts of the subject animal, such as the head, pelvis, the back and at least one limb. Data from each motion sensor is received and processed to provide an indication of lameness in the fore- and/or hindlimbs of the subject animal.

US 2002/0188219 discloses a method and apparatus for inferring physical/mental fitness of a subject through eye response analysis. In particular, the subject is provided with a series of visual stimuli in the form of moving images. The response of the eye of the subject to the moving images is observed and compared with a reference eye pattern. The results of the comparison are used to provide an indication of the level of the fitness of the subject.

A technique has now been found that allows living embryos to be analysed in real time. In particular, it has now been found that a close correlation exists between the movement pattern of a healthy embryo and its stage of development. In particular, it has been found that an embryo exhibits different patterns of movement at different development stages. As a result, it has been found possible to determine the developmental stage of an embryo by analysing its movement. Further, and advantageously, it has been found that the analysis of the movement of an embryo may be automated, avoiding the need for manual observation of the embryos and its attendant problems.

Accordingly, in a first aspect, the present invention provides a method for characterising an embryo, the method comprising:

determining characteristics of the pattern of movement of the embryo; and

comparing the determined characteristics with reference data relating to the expected movement pattern of the subject embryo.

In a further aspect, the present invention provides a system for characterising an embryo, the system comprising:

imaging apparatus for capturing image data of the embryo at a plurality of moments in time;

a device for receiving the image data and determining characteristics of the pattern of movement of the embryo.

The method and system of the present invention obtain data relating to the movement of the embryo under investigation and determine characteristics of the pattern of movement of the embryo. The data relating to the characteristics are compared with data relating to the known or expected pattern of movement, allowing the user to characterise the embryo. The characterising of embryos in this way has a number of applications and uses, as discussed in more detail below.

The method and system of the present invention may be applied to any living embryo that can be imaged. For example, organisms that may be analysed using the present invention include those invertebrates, such as molluscs, crustaceans and polychaetes, and vertebrates having free living embryos or embryos that are contained within an egg capsule or sack. Many encapsulated embryos are visible through the wall of the egg capsule or sack, such as many crustaceans, for example lobsters and crabs, molluscs and vertebrates such as fish. Further, with suitable imaging techniques, the present invention may be applied to embryos contained in opaque egg capsules or sacks, such as higher vertebrates. Still further, the present invention is applicable, again with suitable imaging techniques, to the embryos of higher organisms, in particular mammals.

The method and system of the present invention require that image data of the embryo under investigation is gathered. Image analysis and pattern recognition techniques have been used in the identification of animal species. Such techniques are described, for example, Yu, D. S., et al. ‘Identification of ichneumonid wasps using image analysis of wings’, systematic Entomology 17 (1992), pages 389 to 395; Dorge, T. et al., ‘Direct identification of pure Penicillium species using image analysis’, Journal of Microbiological Methods, 41 (2000), pages 121 to 133; and Schroder, S. D., et al., ‘The new key to bees: automated identification by image analysis of wings’, In, Kevan, P. and Imperatriz Fonseca, V. L. (Eds), ‘Pollinating bees—the conservation link between agriculture and nature’, Ministry of Environment, Brasilia, (2002) pages 209 to 216. However, none of these techniques employ an analysis of movement of the subject under investigation.

Any suitable imaging means may be employed. The type of imaging means will be determined, at least in part, by the nature of the subject embryos and their surroundings. For free living embryos and embryos visible through the wall of the egg capsule or sack, the imaging means may be photographic or video imaging system. For embryos that are not visible in their surroundings, other imaging means are employed, for example ultrasound or infrared imaging systems. Suitable imaging means for both visible and non-visible embryos are known in the art and are commercially available.

The present invention requires image data of the embryo to be collected over a period of time, to allow characteristics of the movement patterns of the embryos to be determined. The image data may be continuous image data, for example video data. Alternatively, the image data may comprise a plurality of still images of the embryo taken at intervals over the time period of the observation. The image data of the present invention are preferably collected in real time, so as to provide a recording of the movement of the embryo in real time.

The embryos should be observed and image data collected over a sufficiently long period of time for the relevant movement characteristics to be determined. The length of the time period may vary, for example according to the extent or speed of movement of the embryo, with faster moving embryos typically requiring less time for the observation and the required image data to be collected. The imaging time period may range from fractions of a second to tens of seconds, depending upon the embryo being observed and the nature and type of the movement being monitored. In particular, it is preferred that the imaging time period is long enough to allow analysis of movement patterns of the embryo at different frequencies of movement. Typical imaging time periods are from 0.1 second to 20 minutes, more preferably from 0.2 seconds to 10 minutes, still more preferably from 0.5 seconds to 5 minutes. An imaging period of from 30 seconds to 10 minutes is particularly preferred for the analysis of a wide range of embryos.

The image data from observing the movement of the embryos may collected at any suitable frequency. The frequency will depend upon such factors as the nature and type of movement being monitored.

Preferably, the embryos are imaged at a frequency of at least 1 Hz, more preferably at least 2 Hz, still more preferably at least 5 Hz. The embryos may be imaged at a frequency of up to 100 Hz, more preferably up to 75 Hz, still more preferably up to 50 Hz. An imaging frequency in the range of from 1 to 100 Hz, more preferably from 2 to 75 Hz, still more preferably in the range of from 3 to 50 Hz is suitable for many embryo movement patterns. For example, for imaging movement of the entire embryo, such as rotation or displacement, an imaging frequency of from 3 to 25 Hz, more preferably from 5 to 20 Hz is suitable. A particularly preferred imaging frequency in this case is from about 5 to 15 Hz. For collecting image data for movements of parts of the embryo, such as movement of muscle tissue, movement of cilia or the flick of a tail, a higher imaging frequency may be required, for example from 25 to 50 Hz.

The frequency of imaging of the embryos may also be expressed in terms of frames per second (fps) of the imaging apparatus. Again, as noted above, the number of frames per second employed in the imaging step will depend upon such factors as the movement pattern of the embryos under analysis and the frequency of movement. The imaging step may be conducted at a speed of from 1 to 50 fps, preferably from 2 to 40 fps, more preferably from 5 to 35 fps. In one embodiment, the imaging apparatus is operated at a speed of from 7.5 to 30 fps.

The embryo may be imaged continuously or at intervals, as noted above. In the latter case, the interval is preferably consistent with Shannon's sampling theorem, which determines the lower limit of sampling interval to avoid aliasing, consistent with observing a maximum rate of change in object motion in a set of consecutive images. Reference in this respect is made to C. E. Shannon, ‘Communication in the presence of noise’, Proc. Institute of Radio Engineers, vol. 37, no. 1, pages 10-21, January 1949; reprinted as a classic paper in Proc. IEEE, vol. 86, no. 2, (February 1998).

The movement of the embryo being observed may be any suitable movement of the embryo or a part thereof. For example, the observed movement may be on a large scale and may be that of the entire embryo, such as the displacement movement of the embryo or its rotational movement. Alternatively, the observed movement may be on a small scale and be of a part of the embryo, for example regions of muscle mass and/or cilia of the embryo. The time period of the observation is selected according to the movement or motion being observed and/or its frequency.

In a preferred embodiment, the observed movement of the embryo is on a first, large scale, preferably movement of the entire embryo, and on a second, small scale, preferably movement of a part of the embryo, such as small scale muscle movements. It has been found that observation of the movement of the embryo on both the aforementioned large scales and small scales provides a particularly comprehensive set of data for further characterising the embryo. Observing the movement of an embryo on both of such large and small scales could not be achieved using manual observation.

The smallest observable movement depends only on the image pixel resolution. Therefore, to reduce the observable scale, and view smaller displacement movements of part or the whole of the embryo, it is possible to increase the image pixel size (for example for 4096 by 2048 to 8192 by 4096 pixels).

Once collected, the image data are analysed to determine characteristics of the movement pattern and of the embryo. The embryo and its movement may be characterised using any suitable parameters. In one preferred embodiment, the image data are analysed to determine parameters of the rotation of the embryo, for example the rate of rotation of the embryo and the mean angles of rotation of the embryo, in particular the mean angles of positive and negative rotation of the embryo. The mean angles of rotation may be used to determine the centre of mass and the centre of rotation of the embryo. One preferred movement parameter to determine is the movement of the centre of mass of the embryo, in particular to determine the extent of rotational movement and/or linear movement of the centre of mass.

Further analysis of the movement pattern may be carried out, to determine the distribution of energy of the embryo with respect to time. Suitable techniques for conducting this form of analysis are known in the art and include Fourier analysis. In particular, Fourier analysis allows oscillatory motion of the embryos to be studied.

Further, as noted above, the movement analysis may be conducted on the whole embryo or a part thereof. For example, the movement analysis may be directed to the motion of nascent organs, to determine their state of development, function and the like. Such movement analysis may also be employed to monitor the development of the organs of the embryo, for example to identify unhealthy or deformed embryos.

The movement of the embryo may be determined using any suitable coordinate basis. For example, the movement may be characterised using rectangular coordinates, to provide movement in terms of orthogonal X and Y directions. Alternatively, the movement may be characterised in terms of radial or polar coordinates, in which the radius (Rho) from a centre of measurement and the angle (Theta) may be determined.

Further, the path the embryo takes as it moves may be analysed in time. The Fourier process may be used to convert a time-sequence of such movement into a frequency representation and repetitive or oscillatory motions are then indicated as repetitions measured in Hertz and with a magnitude. In this case, the term Rho is the radius of the coordinate, Theta the angle at that radius. The intersection of the angle Theta at radius Rho gives a precise position that can be interpreted as an X,Y coordinate.

It has been found that the characterisation of the movement pattern of an embryo when using movement image data collected at significantly different time periods and on significantly different scales, as described hereinbefore, requires specific mathematical analysis. In particular, it has been found that Fourier analysis of such a wide range of movement data is especially advantageous.

Fourier analysis allows the spectral distribution of parameters of the motion of the embryos to be generated. This technique is particularly advantageous, as it allows data to be generated on the movement patterns of embryos moving with different frequencies. The data thus generated may then be used in multidimensional scaling, in turn allowing the frequency distributions of embryo motion patterns to be generated. Such frequency distributions may be used in grouping embryos with particular movement patterns.

The processing of the collected image data may be carried out using any suitable device or processor, with suitable processing systems being known in the art and commercially available. The processing system preferably has a data storage facility or memory for storing data, in particular data relating to the movement of the subject embryos, such as data relating to the movement patterns of the embryo at different developmental stages and/or the normal expected movement pattern of the embryos. In this way, data may be retrieved from the memory, for example to allow a comparison between the data relating to the movement of the subject embryo and stored data determined at an earlier time in the development of the embryo or data gathered from the analysis of other embryos of the same or related species.

The measured or determined characteristics of the movement pattern of the embryo are used to characterise the embryos. The data relating to the movement of the embryo generated by the analysis of the image data may be provided to a storage device, for later retrieval and use. The data may also be displayed in any suitable format on a visual display device. Suitable display devices are known in the art and are commercially available. Suitable formats for the display of the data generated and the characteristics of the embryo include graphical and tabulated data displays.

A range of characteristics of the embryos may be determined from the analysis of their movement patterns. This in turn provides a number of different applications and uses for the method and system of the present invention.

It has been found that the movement patterns of the embryos vary according to the developmental stage of the embryo. Accordingly, the method and system of the present invention may be applied in staging embryos, that is to determine their stage of development.

Therefore, in a further aspect, there is provided a method for staging a subject embryo, the method comprising:

determining characteristics of the pattern of movement of the subject embryo;

comparing the determined characteristics with reference data relating to the expected movement pattern of the subject embryo at different developmental stages; and

using the comparison to determine the stage of development of the subject embryo.

In a further aspect, the present invention provides a system for staging a subject embryo, the system comprising:

imaging apparatus for capturing image data of the subject embryo at a plurality of moments in time;

a processor for receiving the image data and determining characteristics of the pattern of movement of the subject embryo;

a data storage device for storing data relating to the movement patterns of the embryo at different developmental stages; and

a processor for comparing the characteristics of the pattern of movement of the subject embryo with data retrieved from the data storage device, to provide an indication of the developmental stage of the subject embryo.

The method and system of this aspect of the present invention compare data characterising the movement pattern of the subject embryo with existing data retrieved from a data storage device, to provide a determination of the stage of development of the embryo. The analysis of the image data and the comparison of the movement characterising data may be performed by the same or different processors.

It has further been found that the movement pattern of an embryo varies according to aspects of the environment of the embryo. In particular, it has been found that the movement pattern of a challenged and stressed embryo is different to the movement pattern of an embryo that is not challenged. Challenges to the embryo may arise from conditions prevailing in the surroundings of the embryo, for example the ambient conditions, such as temperature and pressure and oxygen concentration. More importantly, challenges to the embryo may arise from the presence of pollutants or toxins in the environment of the embryo. Accordingly, analysis of the movement patterns of the subject embryo using the techniques of the present invention may also be used to provide an indication of the conditions prevailing in the surroundings to the embryo, in particular the presence of pollutants or toxins in the immediate environment.

Therefore, in a further aspect, the present invention provides a method of analysing an environment, the method comprising:

determining characteristics of the pattern of movement of an embryo exposed to the environment; and

comparing the determined characteristics with reference data relating to the expected movement pattern of the subject embryo in the environment.

In a further aspect, the present invention provides a system for analysing an environment, the system comprising:

imaging apparatus for capturing image data of an embryo exposed to the environment at a plurality of moments in time;

a processor for receiving the image data and determining characteristics of the pattern of movement of the embryo;

a data storage device for storing data relating to the normal movement patterns of the embryo; and

a processor for comparing the characteristics of the pattern of movement of the subject embryo with data retrieved from the data storage device.

The movement of the embryo may be analysed while the embryo is being exposed to the environment being analysed. Alternatively, the embryo may be removed from the environment and its movement analysed during the period of time that any stresses to the embryo arising from the environment remain effective.

In one embodiment, this aspect of the invention is used to analyse the presence or absence of chemical stressors, such as pollutants and/or toxins in an environment. For example, the invention may be used to measure or monitor the presence of water-borne and/or air-borne contaminants, pollutants or toxins. Similarly, the invention may be used to determine when critical chemicals, such as oxygen, are in low concentration or absent from the environment of the embryo. Further, the invention may be used to obtain an indication of the exposure of the embryos to radiation, such as ultraviolet radiation and the like.

The embryos may be those of organisms naturally occurring in the environment being analysed, the embryos being removed from the environment or analysed in situ. Alternatively, the embryos may be placed in or exposed to the environment being analysed and either removed after a suitable period of time or analysed in situ. In the case that specific contaminants, pollutants or toxins are being targeted, it is preferred that the embryos are selected to be responsive to challenge by the target chemicals. Alternatively, embryos may analysed to gain an overall indication of the level or contamination or pollution of a particular environment.

In particular, it has been found that the movement patterns of water-borne embryos exhibit significant variations when the embryos are challenged by toxins present in the water. To be effective, a technique for environmental monitoring requires rapid, high through-put biological assays. In this way, the present invention provides an effective way of monitoring the quality of water, such as the water in rivers, streams, ponds, lakes, seas and oceans. Suitable organisms for use in such methods include, but are not limited to, crustaceans, such as shrimps, lobsters, crabs, molluscs and fish. Suitable organisms further include species associated with both the plankton, that is species living in the water column, and the benthos, that is species living bottom sediments.

In a similar way, the method and system could be used to test substances to assess their possible environmental impact, by determining the response of embryos to the presence of the substances and the level of stress induced in the embryos when so challenged. More generally, the techniques of the present invention may be used to analyse the toxicological effects of new chemicals, including pharmaceuticals.

Embodiments of the present invention will now be described, for illustrative purposes only, by way of the following examples. Data generated during the experiments described in the examples are set out in the accompanying figures, details of which are provided in the relevant example.

### EXAMPLES

### Example 1

An experiment was conducted to investigate the movement patterns of pulmonate snail embryos. Six embryos from each of the five developmental stages (trochophore, veliger, early-, mid- and late-hippo) were selected and analysed.

Images of the movement of each subject embryo were recorded for a period of ten minutes. The imaging apparatus comprised a 4 megapixel monochrome camera (AVT Pike 421) operating at 7.5 Hz with a 1 megapixel region of interest, connected to a zooming optic lens system (Zoom 70 XL Optical System with an iris diaphragm) providing 140× magnification.

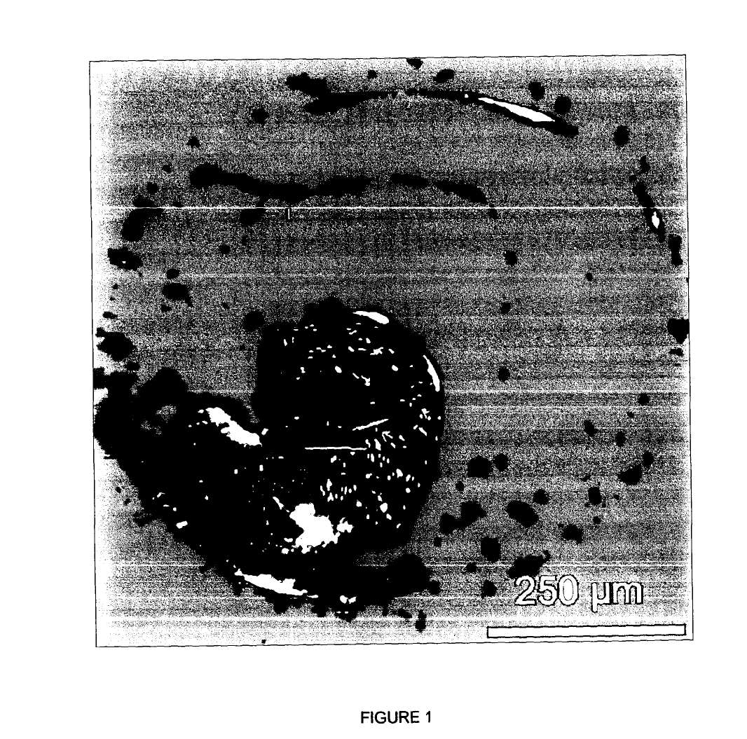

A representative frame of the image data recorded is shown in FIG. 1.

Analysis of the image data was carried out for optical flow using the OpenCV toolkit (royalty free software for commercial applications) and applying the sparse optic flow algorithms described in ‘An iterative image registration technique with an application to stereo vision.’, Lucas, B. D., et al., Proceedings of the 1981 DARPA Imaging Understanding Workshop, pages 121 to 130. This temporal tracking technique first extracts corner features from each image. Thereafter, image features are registered from frame to frame and assigned a velocity variance to each. A feature with a high velocity variance was rejected, as indicating inaccurate tracking. The remaining features were used in subsequent analysis of the movement pattern of the embryo.

Three parameters of motion of the subject embryo were extracted from each frame of the image data and compared with the corresponding parameters in the preceding frames. The three parameters of motion were positive and negative angles of rotation, and Rho angular changes to the centre of mass for the embryo. The centre of mass of the embryo was determined by determining the X and Y coordinates of every point of motion detected. The X and Y coordinates were summed to provide mean values for X and Y.

The resulting data were analysed for spectral content using the Fast Discrete Fourier Transform (FDFT), to generate data relating to the frequency of motion of the subject embryo. By sampling the image data at a rate of 7.5 Hz over a period of approximately 10 minutes for each subject embryo, 4498 image data samples for each embryo were obtained. The frequency analysis revealed repetitive oscillations in the movement of the subject embryos from 10 minutes to 3.75 Hz. 18 frequency bins were analysed from the FDFT analysis, as follows:

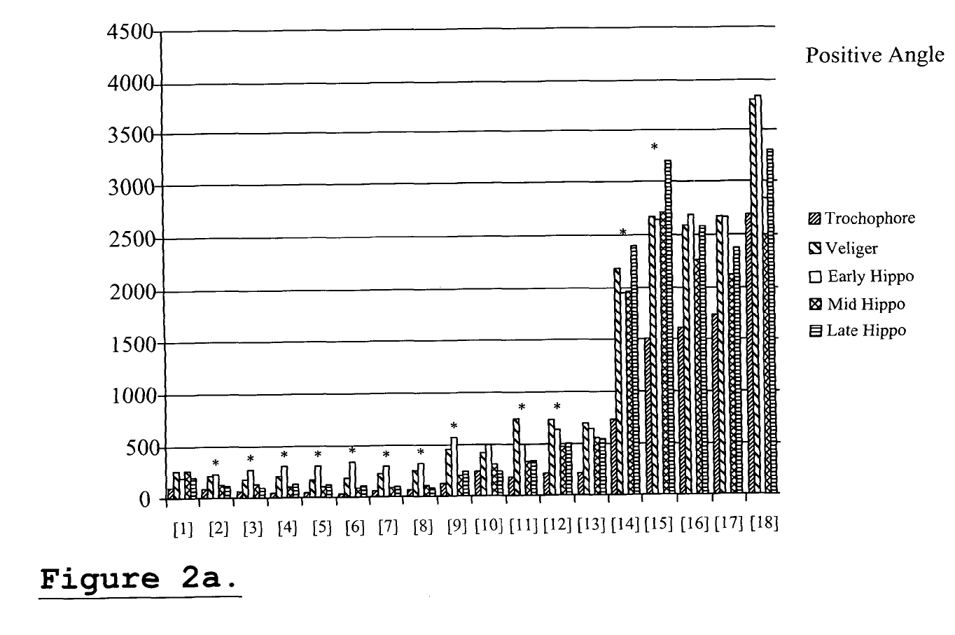

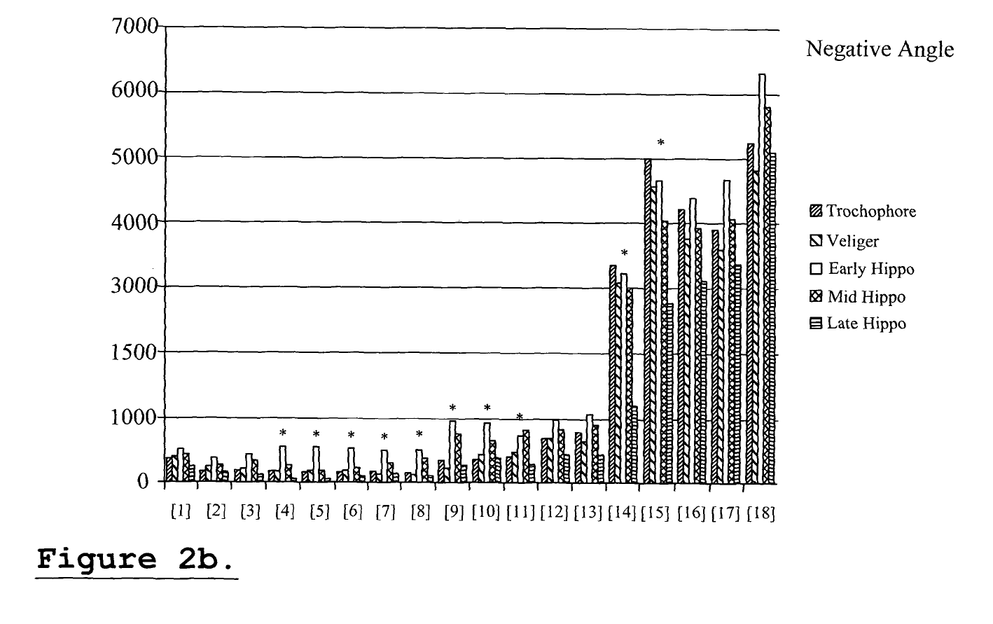

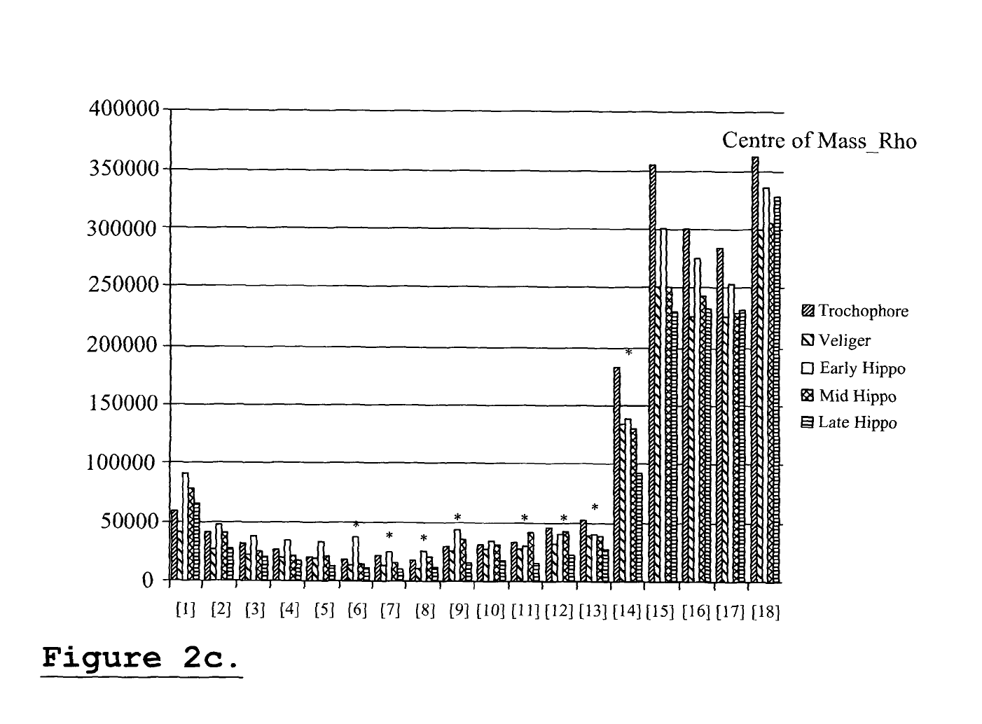

The distribution of energy within each of the 18 frequency bins for each of the three parameters of movement of the subject embryos in each developmental stage are summarised in FIGS. 2a, 2b and 2c. The results indicated with * indicate significant differences in the energy distribution between embryo developmental stages for that wavelength bin.

The results indicated that embryos as the three earliest developmental stages, that is trochophore, veliger and early hippo, exhibit ciliary rotation, indicated by positive and negative angles of rotation. The pattern of positive and negative angles of rotation became less regular as development progressed to the mid-hippo and late-hippo stages, as the embryos developed from ciliary rotation to muscular movement.

The movement of the centre of mass of the embryos in the trochophore developmental stage is a figure of eight pattern, producing a sinusoidal pattern in the X and Y coordinates of the centre of mass. Embryos in the veliger developmental stage exhibited movement of the centre of mass having a frequency close to that of the embryo rotational movement. Early hippo stage embryos exhibited variable rates of rotation, leading to a less uniform movement pattern. Mid-hippo stage embryos exhibit prolonged periods of inactivity, during which the embryo performs muscular contractions and flexing of the foot and shell. Movement of the centre of mass is less rotational and is mostly linear. Embryos in the late-hippo stage exhibit similar movement patterns to those of the mid-hippo stage embryos, but limited to a smaller scale.

The experimental data demonstrated that specific parameters of the movement of the embryos can be identified which vary consistently between the developmental stages. This in turn allows the developmental stage of the embryo to be determined from the analysis of its movement.

### Example 2

The general procedure described in Example 1 was repeated to analyse the movement patterns of pulmonate snail embryos subjected to varying degrees of challenge.

Three saline solutions (1 ppt; 7.5 ppt; and 15 ppt) were prepared and the pulmonate snail embryos divided equally between the solutions. The embryos were placed in the solution for 10 minutes, after which the imaging procedure was started.

Four parameters of motion of the subject embryo were extracted from each frame of the image data and compared with the corresponding parameters in the preceding frames. The four parameters of motion were positive and negative angles of rotation, and rho and theta angular changes to the centre of mass for the embryo.

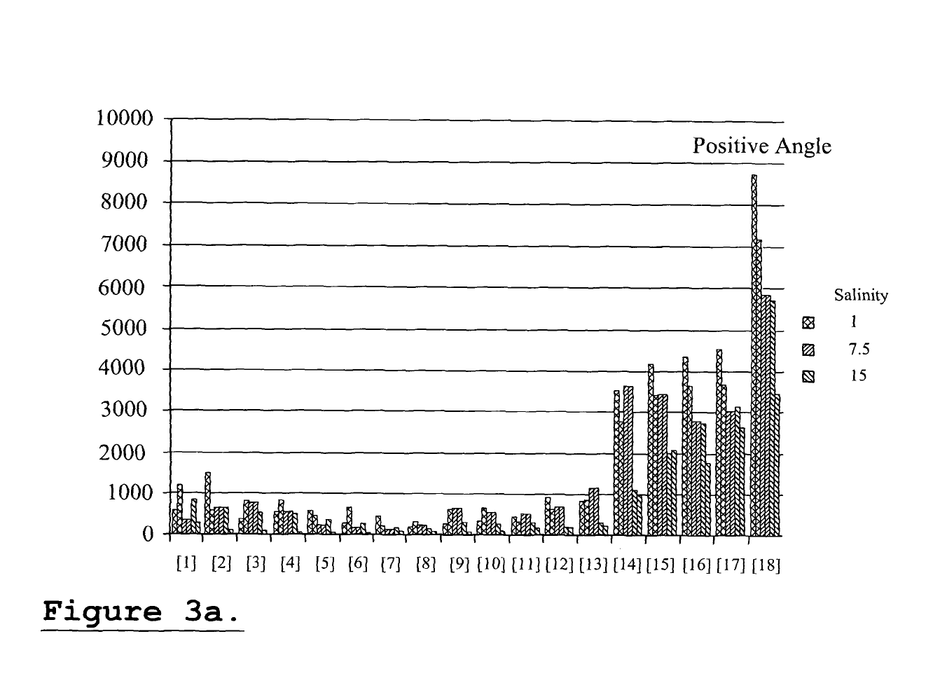

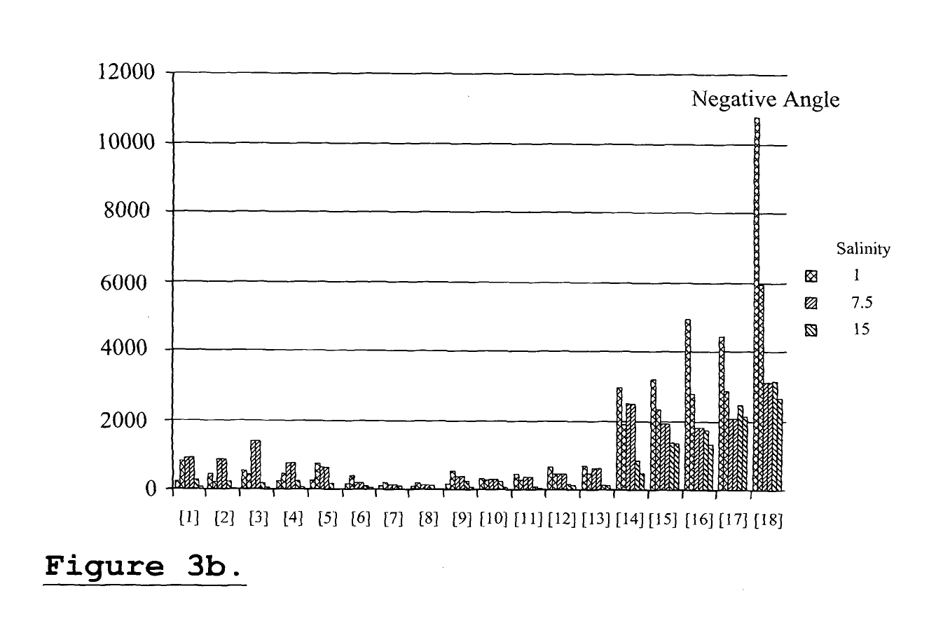

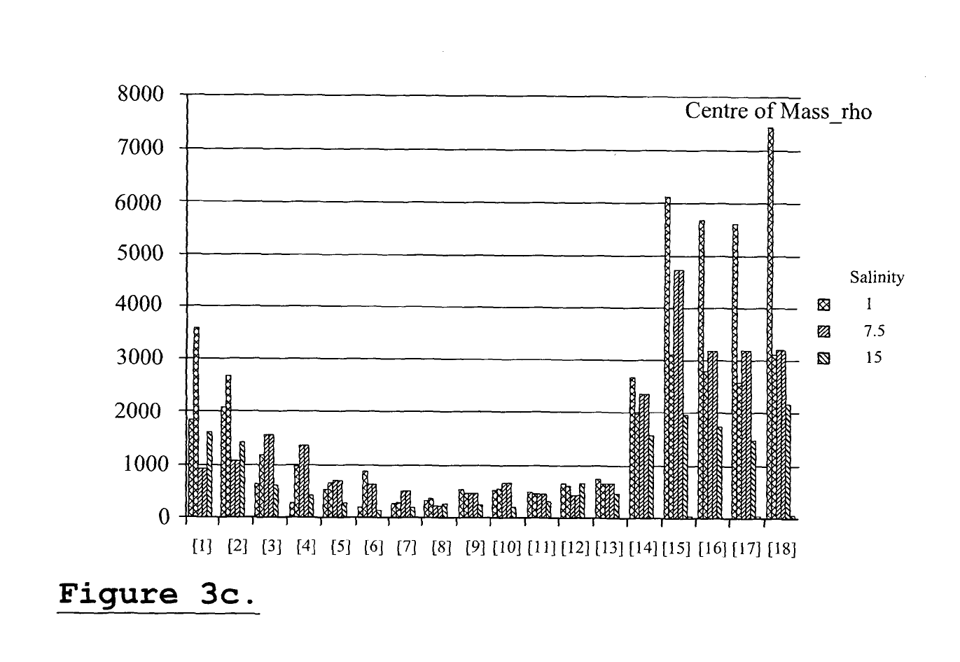

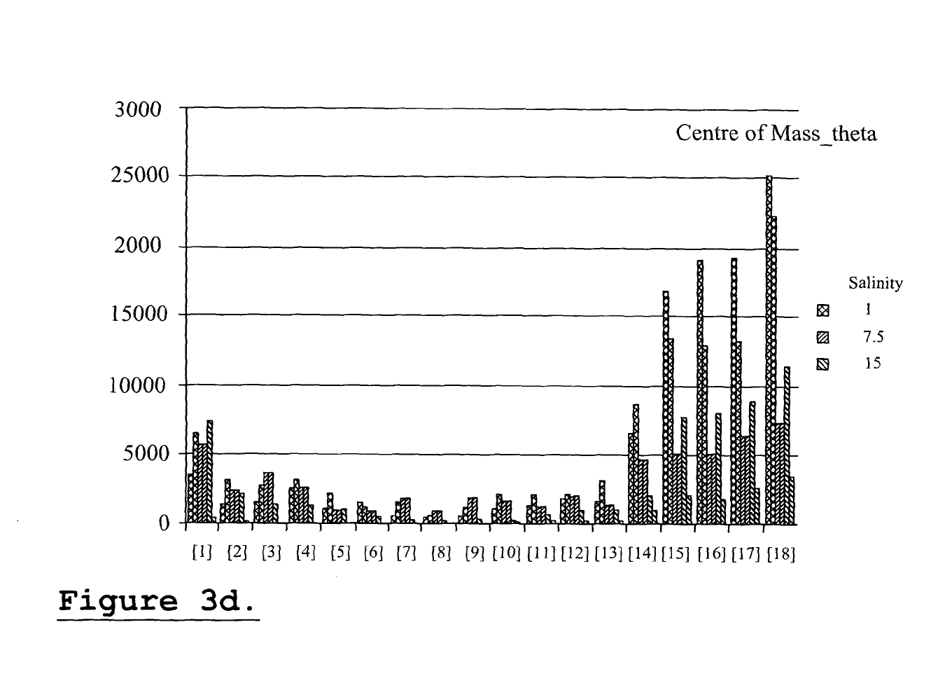

The distribution of energy within each of the 18 wavelength bins for each of the four parameters of movement of the subject embryos in each saline solution are summarised in FIGS. 3a, 3b, 3c and 3d.

The results indicate a significant difference in the derived movement parameters of the embryos between the three saline solutions, confirming that analysis of the movement patterns of the embryos allows the degree of challenge to the embryos by substances in their environment to be determined. This in turn allows the nature and concentration of challenging substances in the environment to be quantified.

A similar result may be achieved when analysing the embryo movement in response to other environmental challenges, such as radiation.

It is to be understood that the techniques described in the above examples may be applied to parts of the embryos, for example particular organs or groups of organs, or a discrete portion of the embryo structure, with similar effect.

### Example 3

An experiment was conducted to investigate the movement patterns of zebra fish (Danio rerio) embryos. Six embryos from each of the three developmental stages (19, 21.5 and 33 hpf) were selected and analysed.

Images of the movement of each subject embryo were recorded for a period of ten minutes. The imaging apparatus comprised a 4 megapixel monochrome camera (AVT Pike 421) operating at 15 Hz with a 1600×1200 pixel region of interest, connected to a zooming optic lens system (Keyence VHZ100R).

Analysis of the image data was carried out for optical flow using the OpenCV toolkit (royalty free software for commercial applications) and applying the sparse optic flow algorithms described in ‘An iterative image registration technique with an application to stereo vision.’, Lucas, B. D., et al., Proceedings of the 1981 DARPA Imaging Understanding Workshop, pages 121 to 130. This temporal tracking technique first extracts corner features from each image. Thereafter, image features are registered from frame to frame and assigned a velocity variance to each, A feature with a high velocity variance was rejected, as indicating inaccurate tracking. The remaining features were used in subsequent analysis of the movement pattern of the embryo.

Four parameters of motion of the subject embryo were extracted from each frame of the image data and compared with the corresponding parameters in the preceding frames. The four parameters of motion were positive and negative angles of rotation, and Rho-theta angular changes to the centre of mass for the embryo. The centre of mass of the embryo was determined by determining the X and Y coordinates of every point of motion detected. The X and Y coordinates were summed to provide mean values for X and Y.

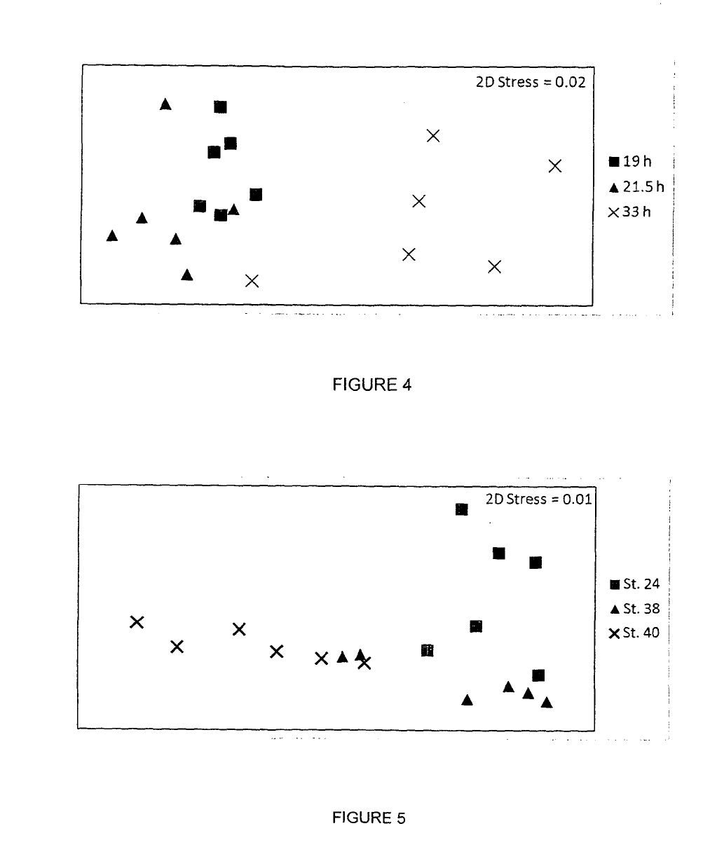

The resulting data were analysed for spectral content using the Fast Discrete Fourier Transform (FDFT), to generate data relating to the frequency of motion of the subject embryo. By sampling the image data at a rate of 15 Hz over a period of approximately 10 minutes for each subject embryo, the frequency analysis revealed repetitive oscillations in the movement of the subject embryos from 10 minutes to 7.5 Hz. 18 frequency bins were analysed from the FDFT analysis. These data were logarithmically transformed (LogX+1) for each embryo and a Bray and Curtis similarity matrix was calculated. This matrix was in turn used to generate multidimensional scaling (MDS) plots and analysis of similarities (ANOSIM) was used to determine the degree of similarity within groups of embryos from particular developmental stages. The MDS plot is shown in FIG. 4.

The embryos as 19 hours post fertilisation (hpf) performed regular movements in the form of flicks of the distal end of the tail of the embryo. This movement resulted in calculated peaks in the positive and negative rotational movement and in X and Y displacement of the centre of mass of the embryo. The embryos at 21.5 hpf performed more infrequent movements of the tail, but movement involved a greater length of the tail. Embryos at 33 hpf displayed longer periods of rest without movement. Movements of these embryos involved more vigorous flicks of the tail, producing larger peaks in the calculated negative and positive movements. X and Y displacement of the embryos at 33 hpf was reduced, due to limited space for the embryo within the egg capsule.

The experimental data demonstrated that specific parameters of the movement of the embryos can be identified which vary consistently between the developmental stages. This in turn allows the developmental stage of the embryo to be determined from the analysis of its movement.

### Example 4

An experiment was conducted to investigate the movement patterns of African clawed frog (Xenopus laevis) embryos. Six embryos from each of three developmental stages (24, 38 and 40 hpf) were selected and analysed.

Images of the movement of each subject embryo were recorded for a period of ten minutes. The imaging apparatus comprised a 4 megapixel monochrome camera (AVT Pike 421) operating at 15 Hz with a 1600×1200 pixel region of interest, connected to a zooming optic lens system (Keyence VHZ20R).

Analysis of the image data was carried out for optical flow using the OpenCV toolkit (royalty free software for commercial applications) and applying the sparse optic flow algorithms described in ‘An iterative image registration technique with an application to stereo vision.’, Lucas, B. D., et al., Proceedings of the 1981 DARPA Imaging Understanding Workshop, pages 121 to 130. This temporal tracking technique first extracts corner features from each image. Thereafter, image features are registered from frame to frame and assigned a velocity variance to each. A feature with a high velocity variance was rejected, as indicating inaccurate tracking. The remaining features were used in subsequent analysis of the movement pattern of the embryo.

Four parameters of motion of the subject embryo were extracted from each frame of the image data and compared with the corresponding parameters in the preceding frames. The four parameters of motion were positive and negative angles of rotation, and Rho-theta angular changes to the centre of mass for the embryo. The centre of mass of the embryo was determined by determining the X and Y coordinates of every point of motion detected. The X and Y coordinates were summed to provide mean values for X and Y.

The resulting data were analysed for spectral content using the Fast Discrete Fourier Transform (FDFT), to generate data relating to the frequency of motion of the subject embryo. By sampling the image data at a rate of 15 Hz over a period of approximately 10 minutes for each subject embryo, the frequency analysis revealed repetitive oscillations in the movement of the subject embryos from 10 minutes to 7.5 Hz. 18 frequency bins were analysed from the FDFT analysis. These data were logarithmically transformed (LogX+1) for each embryo and a Bray and Curtis similarity matrix was calculated. This matrix was in turn used to generate multidimensional scaling (MDS) plots and analysis of similarities (ANOSIM) was used to determine the degree of similarity within groups of embryos from particular developmental stages. The MDS plot is shown in FIG. 5.

The embryos at stage 24 performed occasional flicks of the tail. The movement of the tail was relatively slow, leading to a peak in the negative and positive rotational movements, depending upon the direction of the flick. Embryos at stages 38 and 40 exhibited more regular rapid movements of the tail. This movement resulted in sharp peaks in the negative and positive rotational movements calculated by optical flow. The method also determined blood flow in embryos at stages 38 and 40, producing a level of continuous negative and positive movements, compared with embryos at stage 24. Embryos at stages 38 and 40 could be readily distinguished due to the movement arising from heart beats and blood flow in the stage 40 embryos.

The experimental data demonstrated that specific parameters of the movement of the embryos can be identified which vary consistently between the developmental stages. This in turn allows the developmental stage of the embryo to be determined from the analysis of its movement.

### Example 5

The general procedure described in Example 3 was repeated to analyse the movement patterns of zebra fish (Danio rerio) embryos subjected to varying degrees of challenge.

A first group of embryos at stages 19, 21.5 and 22 hpf were placed separately into 20 ml glass vials containing 1.5% ethanol in deionised water. The embryos were exposed to the ethanol solution for 30 minutes prior to observation of their movement patterns and imaging. A second group of embryos at stages 19, 21.5 and 22 hpf were placed separately into 20 ml glass vials containing 5% saline solution. The embryos were exposed to the saline solution for 60 minutes prior to observation of their movement patterns and imaging. A third group of embryos was transferred to 20 ml glass vials containing deionised water 30 minutes before observation, as a control. All vials were held at a constant temperature of 27° C.

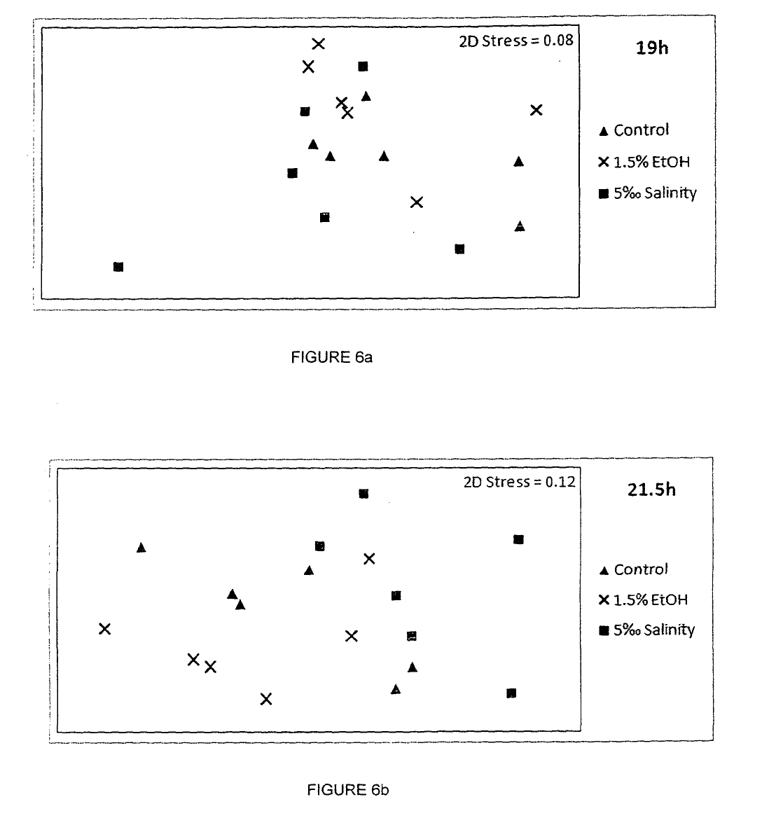

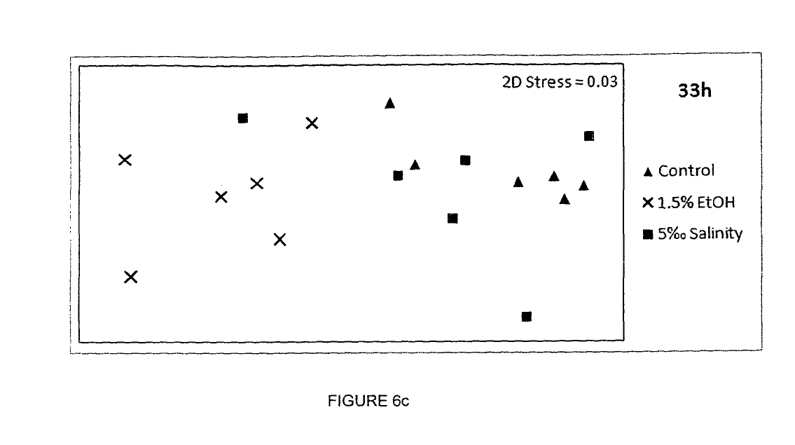

The MDS plots for the embryos at each of the three stages of development are shown in FIGS. 6a to 6c.

The embryos under stress due to exposure to the ethanol solution or the increased salinity were readily identifiable from an observation of their movement patterns and processing of the image data. For example, the embryos at stage 33 exposed to the ethanol solution were readily distinguished from the stage 33 control embryos, in particular as a result of the ANOSIM analysis. Further, stage 21.5 hpf embryos exposed to higher salinity exhibited significantly different movement patterns to those exposed to an ethanol solution. Stage 19 and 21.5 hpf embryos exposed to ethanol were determined by optic flow to have more peaks in positive and negative rotational movement and X and Y displacement of the centre of mass, as a result of an increased frequency of tail flicking, compared with the control embryos.

### Example 6

The general procedure described in Example 4 was repeated to analyse the movement patterns of African clawed frog (Xenopus laevis) embryos subjected to varying degrees of challenge.

A first group of embryos at stages 24, 38 and 40 hpf were placed separately into 20 ml glass vials containing 20% saline solution. The embryos were exposed to the saline solution for 20 minutes prior to observation of their movement patterns and imaging. A third group of embryos was transferred to 20 ml glass vials containing deionised water 20 minutes before observation, as a control. All vials were held at a constant temperature of 23° C.

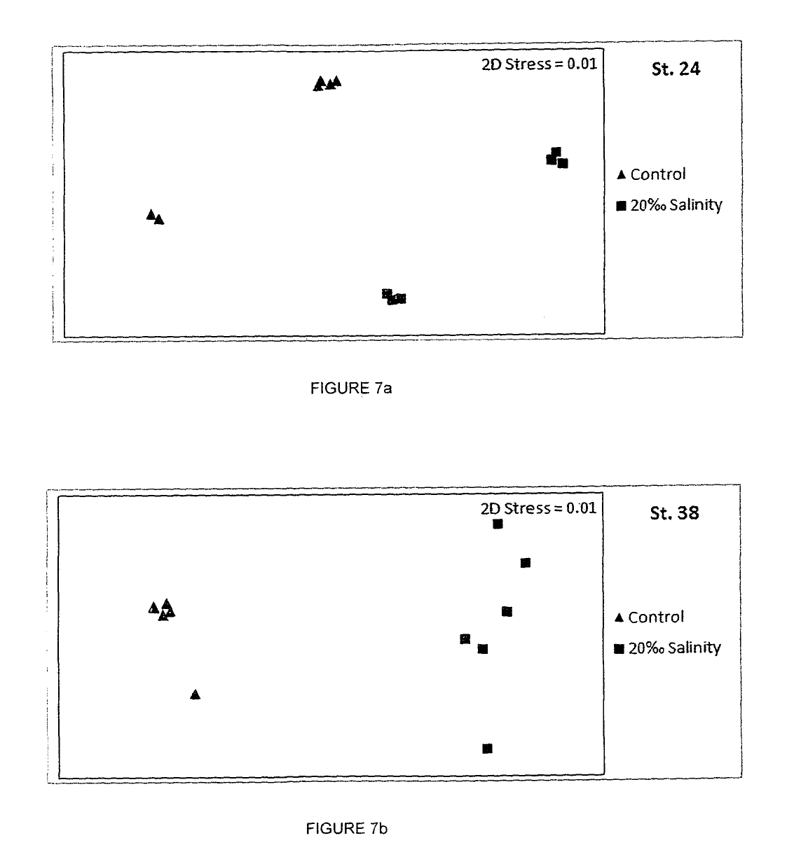

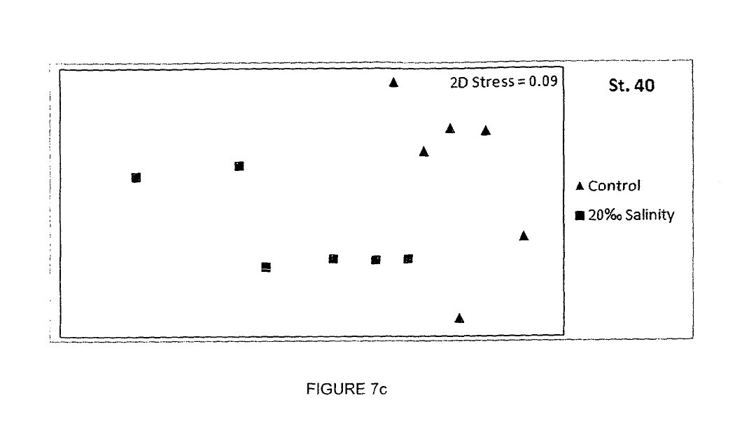

The MDS plots for the embryos at each of the three stages of development are shown in FIGS. 7a to 7c.

The embryos under stress due to exposure to increased salinity were readily identifiable from an observation of their movement patterns and processing of the image data. In particular, the processed image data readily distinguished the embryos at all levels of development exposed to increased salinity from the control embryos, by virtue of increased positive and negative peaks in the optic flow parameters, as a result of increased movement of the tail of the embryos.

### Example 7

The following general procedure was followed to analyse the movement patterns of freshwater snail (Radix balthica) embryos subjected to varying degrees of (salinity stress) challenge.

Embryos at stages E3, E4, E6, E9 and E11 of development were placed in 96 well plates, one embryo for each well. The wells were filled with artificial pond water at one of three salinities: 2.5%; 5%; or 7.5%; or with artificial pond water as a control. The embryos were exposed to the increased salinity for 10 minutes prior to observation and image analysis of movement patterns.

Images of the movement of each subject embryo were recorded for a period of ten minutes. The imaging apparatus comprised a 4 megapixel monochrome camera (AVT Pike 421) operating at 7.5 Hz with a 1000×1000 pixel region of interest, connected to a zooming optic lens system (Zoom 70 XL Optical System).

Analysis of the image data was carried out for optical flow using the OpenCV toolkit (royalty free software for commercial applications) and applying the sparse optic flow algorithms described in ‘An iterative image registration technique with an application to stereo vision.’, Lucas, B. D., et al., Proceedings of the 1981 DARPA Imaging Understanding Workshop, pages 121 to 130. This temporal tracking technique first extracts corner features from each image. Thereafter, image features are registered from frame to frame and assigned a velocity variance to each. A feature with a high velocity variance was rejected, as indicating inaccurate tracking. The remaining features were used in subsequent analysis of the movement pattern of the embryo.

Four parameters of motion of the subject embryo were extracted from each frame of the image data and compared with the corresponding parameters in the preceding frames. The four parameters of motion were positive and negative angles of rotation, and Rho-theta angular changes to the centre of mass for the embryo. The centre of mass of the embryo was determined by determining the X and Y coordinates of every point of motion detected. The X and Y coordinates were summed to provide mean values for X and Y.

The resulting data were analysed for spectral content using the Fast Discrete Fourier Transform (FDFT), to generate data relating to the frequency of motion of the subject embryo. By sampling the image data at a rate of 7.5 Hz over a period of approximately 10 minutes for each subject embryo, the frequency analysis revealed repetitive oscillations in the movement of the subject embryos from 5 minutes to 3.75 Hz. 18 frequency bins were analysed from the FDFT analysis. These data were logarithmically transformed (LogX+1) for each embryo and a Bray and Curtis similarity matrix was calculated. This matrix was in turn used to generate multidimensional scaling (MDS) plots and analysis of similarities (ANOSIM) was used to determine the degree of similarity within groups of embryos from particular developmental stages.

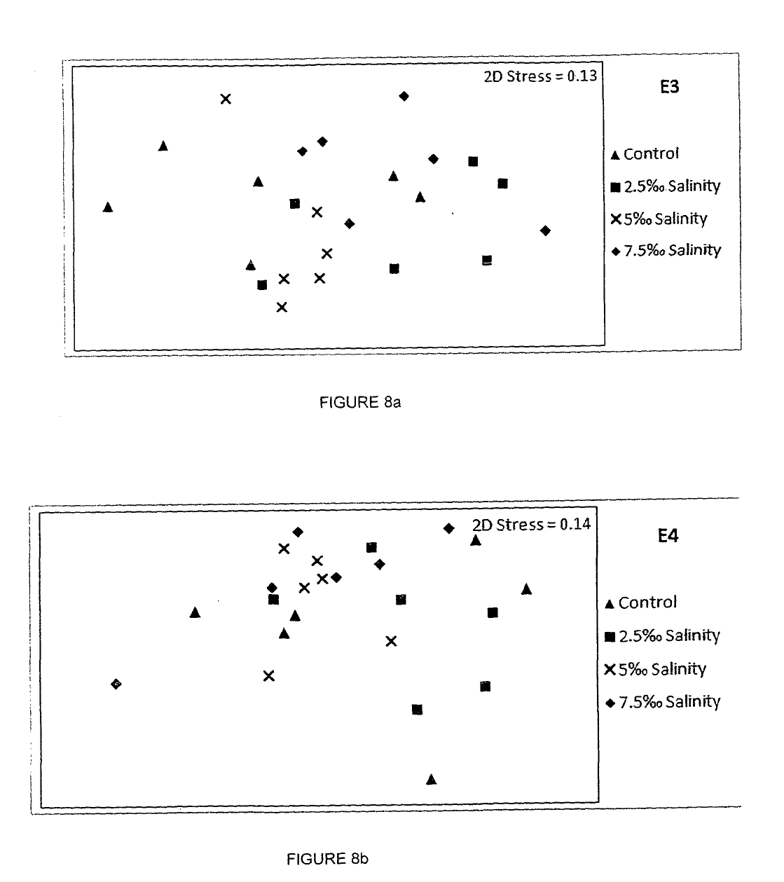

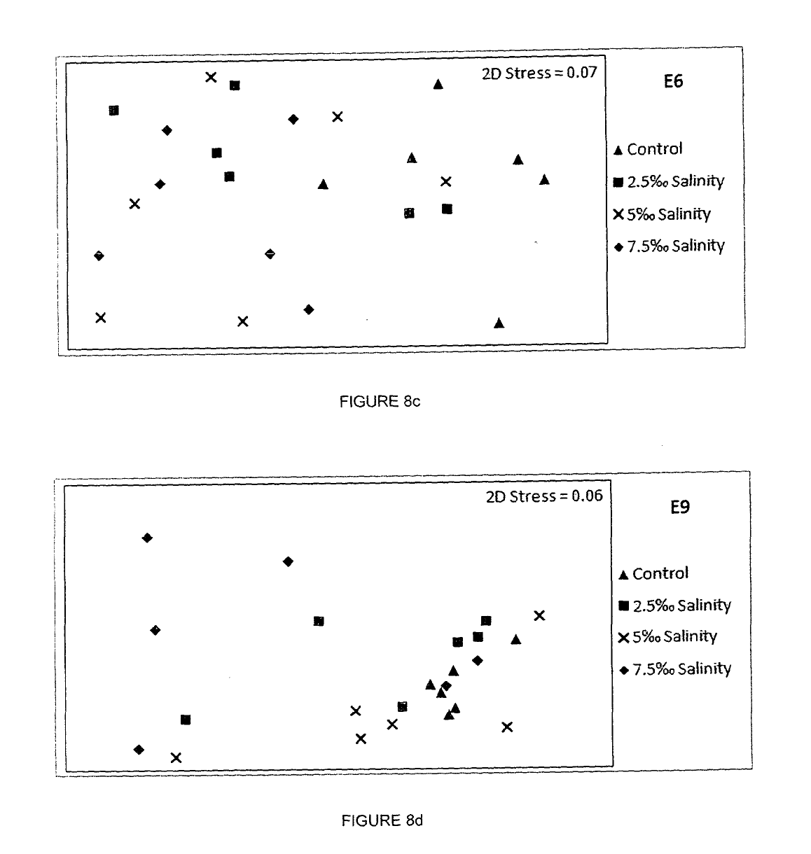

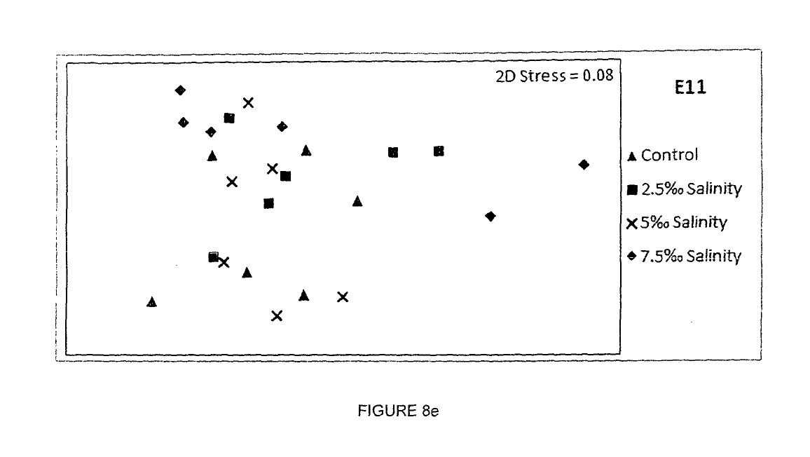

The MDS plots are shown in FIGS. 8a to 8e.

The embryos under stress due to exposure to increased salinity were readily identifiable from an observation of their movement patterns and processing of the image data. In particular, the processed image data readily distinguished the embryos at all levels of development exposed to increased salinity from the control embryos, by virtue of reduced number of peaks in the positive and negative rotational movement and the X and Y displacement of the centre of mass of the embryos, resulting from a general reduction in the movement of the embryos when in the saline solution.

1-28. (canceled)

29. A method for characterising an embryo, the method comprising:
determining characteristics of the pattern of movement of the embryo; and
comparing the determined characteristics with reference data relating to the expected movement pattern of the subject embryo.

30. The method according to claim 25, wherein the characteristics of the pattern of movement of the embryo are determined using imaging of the embryo to collect image data of the embryo and by analysis of the image data so-collected.

31. The method according to claim 26, wherein the image data is collected by photographic imaging, video imaging, ultrasound imaging or infrared imaging.

32. The method according to claim 26, wherein the image data are collected continuously or intermittently.

33. The method according to claim 26, wherein the image data are collected over a period of time of from 1 second to 20 minutes.

34. The method according to claim 25, wherein rotational movement of the embryo is characterised.

35. The method according to claim 30, wherein the mean angle of rotation of the embryo is determined.

36. The method according to claim 31, further comprising determining the centre of mass of the embryo from analysis of the mean angle of rotation.

37. The method according to claim 25, wherein the movement of the embryo is characterised by analysis of the movement of the centre of mass of the embryo.

38. The method according to claim 25, wherein the movement pattern of the embryo is analysed to determine the distribution of energy of the embryo.

39. The method according to claim 25, wherein the movement determined is the movement of at least one organ of the embryo.

40. The method according to claim 25, wherein the movement of the embryo is characterised in terms of oscillatory motions of the embryo.

41. A method for staging a subject embryo, the method comprising:
determining characteristics of the pattern of movement of the subject embryo;
comparing the determined characteristics with reference data relating to the expected movement pattern of the subject embryo at different developmental stages; and
using the comparison to determine the stage of development of the subject embryo.

42. A method of analysing an environment, the method comprising:
determining characteristics of the pattern of movement of an embryo exposed to the environment; and
comparing the determined characteristics with reference data relating to the expected movement pattern of the subject embryo in the environment.

43. The method according to claim 38, wherein the comparison is made with reference data relating to the presence of predetermined chemicals in the environment to determine the concentration of the predetermined chemicals in the environment of the embryo.

44. The method according to claim 39, wherein the embryos are of organisms occurring naturally in the environment being investigated.

45. The method according to claim 39, wherein the embryos are of organisms not occurring naturally in the environment being investigated.

46. The method according to claim 38, wherein the embryos are removed from the environment before being analysed.

47. A system for characterising an embryo, the system comprising:
imaging apparatus for capturing image data of the embryo at a plurality of moments in time;
a device for receiving the image data and determining characteristics of the pattern of movement of the embryo.

48. The system according to claim 43, wherein the imaging apparatus is selected from the group consisting of a photographic imaging apparatus, a video imaging apparatus, an ultrasound imaging apparatus, and an infrared imaging apparatus.

49. The system according to claim 43, wherein the imaging apparatus is operable to capture images intermittently.

50. The system according to claim 43, wherein the imaging apparatus is operable to capture real time image data of the movement of the embryo.

51. A system for staging a subject embryo, the system comprising:
imaging apparatus for capturing image data of the subject embryo at a plurality of moments in time;
a processor for receiving the image data and determining characteristics of the pattern of movement of the subject embryo;
a data storage device for storing data relating to the movement patterns of the embryo at different developmental stages; and
a processor for comparing the characteristics of the pattern of movement of the subject embryo with data retrieved from the data storage device, to provide an indication of the developmental stage of the subject embryo.

52. A system for analysing an environment, the system comprising:
imaging apparatus for capturing image data of an embryo exposed to the environment at a plurality of moments in time;
a processor for receiving the image data and determining characteristics of the pattern of movement of the embryo;
a data storage device for storing data relating to the normal movement patterns of the embryo; and
a processor for comparing the characteristics of the pattern of movement of the subject embryo with data retrieved from the data storage device.
